(19) United States
(12) Patent Application Publication
(10) Pub. No.: US 2024/0264148 A1
(43) Pub. Date: Aug. 8, 2024

(54) SYSTEM AND METHOD OF IDENTIFYING BIOMARKERS FOR CHEMO-RESISTANCE IN CANCER AND IDENTIFYING TARGETS THAT CAN REVERSE CHEMO-RESISTANCE IN CANCER

(71) Applicant: OROC, Bay Village, OH (US)

(72) Inventor: Saubhik SENGUPTA, Kolkata (IN)

(21) Appl. No.: 18/612,978

(22) Filed: Mar. 21, 2024

Related U.S. Application Data
(63) Continuation of application No. 18/359,215, filed on Jul. 26, 2023, now abandoned.
Application No. 18/359,215 is a continuation of application No. 16/835,220, filed on Mar. 30, 2020.
(60) Provisional application No. 63/392,460, filed on Jul. 26, 2022.
(60) Provisional application No. 62/826,746, filed on Mar. 29, 2019.
(60) Provisional application No. 62/826,752, filed on Mar. 29, 2019.

(51) Int. Cl. G01N 33/50; C12N 5/09

(52) U.S. Cl. CPC G01N 33/5011; C12N 5/0693

(57) ABSTRACT

A chemopredictive assay, including culturing cancer cells of interest; exposing the cancer cultures to several chemotherapeutic agents; identifying the most effective chemotherapeutic agent; culturing surviving cancer cells to prepare second cultures; exposing the second cultures to several chemotherapeutic agents; and identifying the most effective chemotherapeutic agent for treating recurrent cancer.

### CROSS-REFERENCE TO RELATED APPLICATIONS

This application is a continuation of U.S. patent application Ser. No. 18/359,215, filed Jul. 26, 2023, which application claims the benefit of U.S. Provisional Patent Application Ser. No. 63/392,460, filed Jul. 26, 2022, the disclosures which are incorporated herein by reference for all purposes.

This application is also a continuation of U.S. patent application Ser. No. 16/835,220, filed Mar. 30, 2020, which application claims the benefit of U.S. Provisional Patent Application Ser. No. 62/826,746, filed Mar. 29, 2019 and U.S. Provisional Patent Application Ser. No. 62/826,752, filed Mar. 29, 2019, the disclosures which are incorporated herein by reference for all purposes.

### BACKGROUND

The present invention relates to predictive assays for chemotherapy, more specifically the present invention relates to predictive assays for screening chemotherapeutic agents for efficacy in the treatment of naive, treated metastatic and recurrent solid tumor cancers (breast, lung, head and neck, thyroid, parathyroid, colon and colorectal, esophageal, gastric, gall bladder, pancreas, lymphomas, ovarian and primary peritoneal, vulvar, vaginal, and cervical, urinary bladder, liver).

Chemotherapy relates to the treatment of cancer with drugs that preferentially kill cancer cells. Typically, the chemotherapeutic agent selective by virtue of having a higher toxicity in cells that divide rapidly, such as cancer cells.

The selection of the correct chemotherapeutic agent for treatment is often of great importance, and may take into consideration factors such as the toxicity of the agent, the type of cancer under treatment, and the type and severity of potential side effects of the selected agent and the data of the available clinical trials.

In addition, a chemotherapeutic agent may be selected for an individual patient based upon the specific genetic and phenotypical characteristics of the patients' tumor. This tailored approach may result in a chemotherapy regimen that is both less toxic and more effective for a given individual. Clinical assays that are used to select a chemotherapeutic agent in this way are referred to as chemopredictive assays.

Chemopredictive assays are typically used to select a first-line chemotherapeutic agent. In some cases cancer will recur after an initial therapy. In such instances a different chemotherapeutic agent is typically selected for an additional treatment regimen, in the belief that the recurring tumors will have developed at least some degree of resistance to the first-line chemotherapeutic agent used previously. Unfortunately, there are currently no clinical tools that can be used to accurately predict the best second-line drug for a particular patient. NCCN guidelines of 2018 strictly prohibit the use of these testing strategies for recurrent cases due to lack of data of efficacy of these tests in second line management.

The present disclosure is directed to a chemopredictive assay useful for the selection of chemotherapeutic agents to treat naive, treated, metastatic and recurrent solid tumor cancers.

The present disclosure also describes inventions related to predictive assays for chemotherapy and, more specifically, to inventions that relate to predictive assays for screening chemotherapeutic agents for efficacy in the treatment of recurrent cancers.

Chemotherapy relates to the treatment of cancer with drugs that preferentially kill cancer cells. Typically, the chemotherapeutic agent selective by virtue of having a higher toxicity in cells that divide rapidly, such as cancer cells.

The selection of the correct chemotherapeutic agent for treatment is often of great importance, and may take into consideration factors such as the toxicity of the agent, the type of cancer under treatment, and the type and severity of potential side effects of the selected agent.

In addition, a chemotherapeutic agent may be selected for an individual patient based upon the specific genetic and phenotypical characteristics of the patients' tumor. This tailored approach may result in a chemotherapy regimen is both less toxic and more effective for a given individual. Clinical assays that are used to select a chemotherapeutic agent in this way are referred to as chemopredictive assays.

Chemopredictive assays are typically used to select a first-line chemotherapeutic agent. In some cases cancer will recur after an initial therapy. In such instances a different chemotherapeutic agent is typically selected for an additional treatment regimen, in the belief that the recurring tumors will have developed at least some degree of resistance to the first-line chemotherapeutic agent used previously. Unfortunately, there are currently no clinical tools that can be used to accurately predict the best second-line drug for a particular patient.

The present disclosure is directed to a chemopredictive assay useful for the selection of chemotherapeutic agents to treat recurrent cancers.

The present disclosure also describes inventions that pertain to cancer and ovarian cancer treatment, and particularly, to inventions that identify biomarkers to predict chemo-resistance in cancer and ovarian cancer. Further, the inventions relate to identification of: (i) biomarkers for predicting up-front carboplatin paclitaxel resistance and (ii) targets that can reverse chemo-resistance in ovarian cancer using an in silico approach.

Surgery followed by Carboplatin and Paclitaxel is the standard chemotherapy protocol for ovarian cancer. However, a sub group of patients are up-front resistant to the disease and the majority suffer from recurrence. An unmet medical need is to identify up-front chemo-resistive patients and to reverse chemo-resistance. Using RNA Seq data from a set of 60 clear cell carcinoma and 247 serous carcinoma patients we have identified a possibility to diagnose up-front chemo-resistance by testing for GSTM3, ATP7B and SOD2 expression levels. We also constructed a signaling network from plasma membrane proteins to carboplatin and paclitaxel related proteins followed by identification of possible pathways that can be inhibited to reduce function of XRCC4, XRCC6 and PMS2 proteins based on the pathway strengths. Our calculations indicated that SRC kinases are the most common factor in pathways leading to Carboplatin resistance that can be neutralized by SRC kinase inhibitors.

Ovarian cancer is the deadliest gynecological malignancy worldwide. The standard treatment for advanced ovarian cancer is cytoreductive surgery followed by platinum-based chemotherapy (1). However, the survival rates are very low, largely because of high incidence of recurrence due to resistance to conventional surgery and genotoxic chemotherapies. First-line chemotherapy with carboplatin and paclitaxel achieves an improved CR; however, recurrence occurs in 25% of patients with early stage disease and more than 80% of patients with advanced disease (2). A majority of advanced ovarian cancer patients experience disease relapse within 2 years of the initial treatment of combination chemotherapy (3). The heterogeneity of tumour cells leads to molecular variations in signalling pathways including oncogene activation, tumour suppressor inactivation and various pro-survival genetic mutations (4). Therefore, chemo-resistance to standard chemotherapy regimen has emerged as a major challenge (5). The current therapeutic regimens are fixed linear protocols, but cancer biology is a highly dynamic system. Adapting a therapeutic strategy using systems biology approach based on temporal and spatial variations in tumour is a futuristic goal in oncology (6).

Ovarian carcinomas comprise a heterogeneous group of neoplasms, the four most common subtypes being serous, endometrioid, clear cell and mucinous (7). in advanced stage, the prognosis of patients with clear cell carcinoma was remarkably poorer than that of patients with serous carcinoma (8-11). In the same review of data from 12 prospective randomized GOG trials, advanced-stage clear cell carcinoma had worse progression-free survival and overall survival compared with advanced-stage serous carcinoma (overall survival HR 1.66, 95% Cl 1.43 to 1.91) (9). Furthermore, in the meta-analysis, advanced-stage clear cell carcinoma showed a higher HR for death than serous carcinoma (HR 1.71, 95% Cl 1.57 to 1.86) (10). This poorer outcome for patients with advanced-stage clear cell carcinoma has been confirmed in a study based on SEER data (12).

In first-line chemotherapy for clear cell carcinoma, the response rate to a combination of paclitaxel plus platinum, which is standard therapy for ovarian carcinoma, is thought to be higher (22%-56%) than that of other platinum-based chemotherapy (11%-27%) (13-16). However, the addition of taxane was not an independent prognostic factor in the MITO-9 study (17), and there was no survival benefit in advanced-stage clear cell carcinoma between patients treated with paclitaxel plus platinum compared with those treated with platinum monotherapy in a large Japanese study (18).

It is therefore clear that carboplatin paclitaxel based chemotherapy for ovarian cancer does not work great in clear cell ovarian cancer in comparison to serous carcinoma. Also, identifying patients with up-front resistance or potential to recur should be a priority while managing ovarian cancer patients by subjecting them to first line chemotherapy of carboplatin and paclitaxel. We hypothesized that the carboplatin and paclitaxel target proteins that lead to chemo-resistance should be overexpressed in clear cell carcinoma when compared to serous carcinoma of the ovary. Carboplatin and paclitaxel mechanism of action in the cells is shown in FIG. 1 (19, 20). Action of the drugs can be divided into several functions, i. Entry of drug into cell, ii. Metabolism of the drug, iii. Detoxification of the drug, iv. Defensive action of the toxic effects of the drug in the cell and v. Action of the drug in the cell leading to cell death.

Recent RNA Seq studies on ovarian cancers identified differentially expressed genes between ovarian clear cell and serous carinomas (21). Studies have also demonstrated changes in RNA expression post chemotherapy in serous ovarian cancers (22). We also hypothesize that plasma membrane proteins of a cell can respond to the drugs entering the cell first and can send signals for increased function of the proteins that would interact with the drugs. Identifying these pathways can lead us to potential off-label drugs that could be used to reverse chemo-resistance against these drugs.

Based on the above hypothesis and following by in silico analysis of RNA Seq data, we have identified proteins that can be potential biomarkers to identify carboplatin and paclitaxel resistance in serous carcinoma of the ovary. We have generated a signalome network from the plasma membrane proteins to the proteins related to carboplatin and paclitaxel function. Using these networks, we can personalize therapy for patients suffering from ovarian cancer.

### BRIEF SUMMARY

The present inventions are directed to chemopredictive assays, where the assay includes culturing cancer tissues of interest; exposing the cancer tissue cultures to several chemotherapeutic agents treated in liver organoids (chemotherapy agents treated in liver organoids potentially generate active molecules in the body, as opposed to the drugs given directly to cancer tissues); identifying the most effective chemotherapeutic agent; culturing surviving cancer cells to prepare second cultures; exposing the second cultures to different tissue organoids created in the laboratory to create a metastatic scenario, followed by challenge with several chemotherapeutic agents treated in liver organoids; and identifying the most effective chemotherapeutic agent for treating recurrent cancer.

The present inventions are also directed to chemopredictive assays, where the assay includes culturing cancer cells of interest; exposing the cancer cultures to several chemotherapeutic agents; identifying the most effective chemotherapeutic agent; culturing surviving cancer cells to prepare second cultures; exposing the second cultures to several chemotherapeutic agents; and identifying the most effective chemotherapeutic agent for treating recurrent cancer.

The present inventions also pertain to cancer and ovarian cancer treatment, and particularly, to inventions that identify biomarkers to predict chemo-resistance in cancer and ovarian cancer. Further, the inventions relate to identification of: (i) biomarkers for predicting up-front carboplatin paclitaxel resistance and (ii) targets that can reverse chemo-resistance in ovarian cancer using an in silico approach.

### BRIEF DESCRIPTION OF THE DRAWINGS

FIG. 1 is a flowchart for a method of screening chemotherapeutics for second-line chemotherapy, according to a representative embodiment of the invention.

FIG. 2 is a flowchart for a method of screening chemotherapeutics for second-line chemotherapy, according to a representative embodiment of the invention.

FIG. 3A-3B are each flow diagrams showing the mechanism of action of carboplatin and paclitaxel, respectively, in a cell.

FIG. 4A-4C are diagrams showing GSTM3, ATP7B and SOD2 in clear cell carcinoma.

FIG. 5A-5Ciii depicted aspects of the gene expression of plasma membrane proteins in Homo sapiens for both clear cell and serous carcinoma of the ovary.

FIG. 6 shows the pathways that are maximally activated for stimulating XRCC4, XRCC6 and PMS2 function that can lead to DNA repair post Carboplatin treatment.

### DETAILED DESCRIPTION

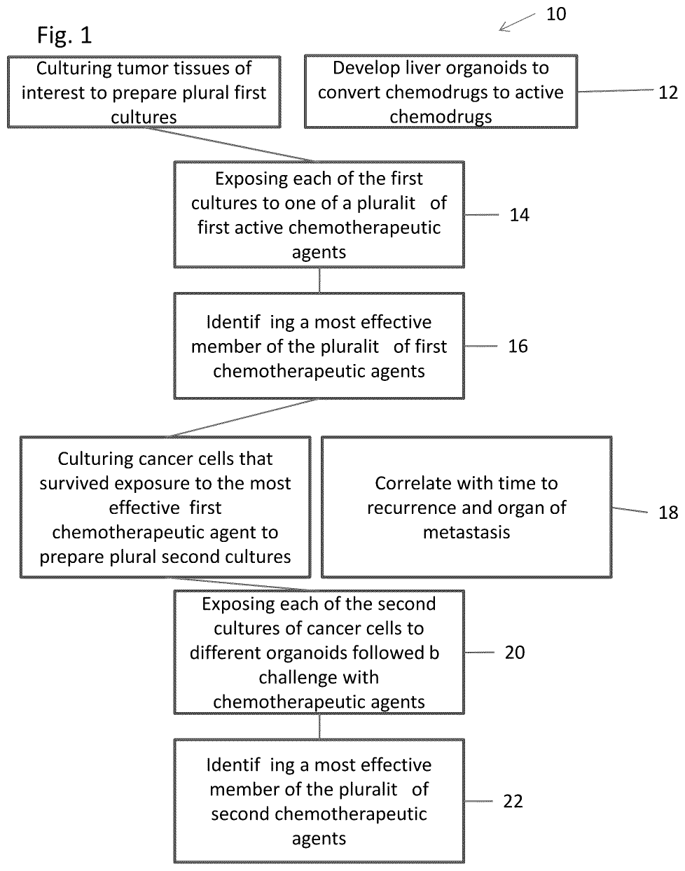

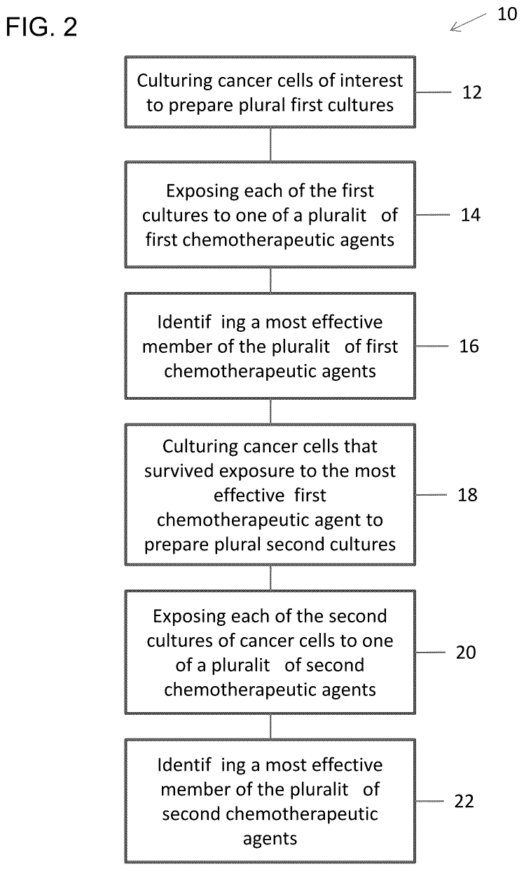

The present chemopredictive assay includes: a) a screening process for chemotherapeutic agents, where the screening process determines the effectiveness of the chemotherapeutic agents against naive, treated metastatic or recurring cancer cells and b) a method to identify target organs of metastasis and time to recurrence. Referring to FIG. 1, a representative example of a method of the present invention is depicted in flowchart 10, and includes culturing liver organoids and exposing each of the chemo-drugs to the liver organoids to generate active drugs at 12, followed by using these drugs to treat tumors of patients at 14, identifying a most effective member of the plurality of first chemotherapeutic agents at 16, culturing cancer cells that survived exposure to the most effective first chemotherapeutic agent to prepare plural second cultures at 18, exposing each of the second cultures of cancer cells to different organoids followed by second chemotherapeutic agents at 20, and identifying a most effective member of the plurality of second chemotherapeutic agents at 22. At 18, time taken by the organoids to home cancer cells will be directly correlated with patients time to recurrence to generate a predictive model for identifying time to recurrence for end stage management, and the organ where cancer cells home fastest will be directly correlated with the patients' metastatic organ, predicting organ of metastasis.

In general, the present assay is performed under conditions selected to mimic the environment in which the cancer cells of interest exist, optionally including an extracellular matrix and/or a monolayer of normal cells upon a selected substrate. In this environment, selected tumor cells are challenged with multiple chemotherapeutic candidate drugs and a first-line selection of chemotherapy agent is performed, for example by direct histopathology.

Subsequent second-line chemotherapeutic selection is performed by assessing the ability of cells that were exposed to the first-line chemotherapeutic agent to grow into secondary colonies, and their ability to grow in the organoid followed by exposure to a second-line chemotherapeutic agent. The second-line selection of chemotherapeutic agent is based upon the ability of the surviving cancer cells to remain viable after exposure to a variety of second-line chemotherapeutic agents.

Substrate. Cell colonies, either of normal cells or of cancer cells, are typically prepared upon some type of supporting substrate. The substrate may be as basic as the surface of a microwell plate. However, the predictive value of the present screening method may be enhanced by preparing a substrate that more closely resembles the environment within the patient.

In one aspect, the substrate includes a matrix, typically an organic matrix. The matrix may be composed of one or more biological polymers. The matrix may include proteins, and may be a solid or semi-solid matrix. In one embodiment, the matrix includes MATRIGEL, a gelatinous mixture of proteins (BD Biosciences) or hydrogel.

The substrate may be further enhanced by preparing an environment of normal cells collected from the vicinity of the collected cancer cells. For example, normal cells may be cultured in order to prepare a substrate that includes at least a monolayer of normal cells.

The chemotherapeutic agents under evaluation in the present screening process may include any agent of interest selected by the physician. Typically the chemotherapeutic agent will be a drug that has been recognized as having efficacy in chemotherapy. In one embodiment of the invention, the chemotherapeutic agents being screened includes one or more of paclitaxel, carboplatin, cisplatin, adriamycin, gemcitabine, topotecan, etoposide, docataxel, ifosamide, and 5-fluoro uracil.

### EXAMPLES

### Example 1. Procurement of Tissue: Sample

A. Tumor/malignant cell sample:
   1. To be collected at the time of core needle biopsy or surgical biopsy, or paracentesis/ascites samples in 10 ml RPMI 1640 medium (without FBS, Penicillin-Streptomycin).
   II. Ascites sample: Ascites collection bottle containing peritoneal washing in 0.9% NaCl solution (Normal Saline) will be taken. This will be transported to the laboratory.
   III. Solid tumor sample will be taken with a new sterile blade.
B. 10 ml whole blood in clotted vial will be collected from the ante-cubital vein.
Collection media: 50 ml sterile glass tubes containing 10 ml of RPMI 1640 medium without fetal bovine serum).
Temperature: Normal ambient temperature.
Sample rejection criteria:
   Smelly or infected samples will be discarded.
   Samples not put into the sterile tube during the process of biopsy will be discarded.
   Sample not resected with fresh sterile blade will be discarded.
   Patient suffering from any viral infections during the time of surgery (even in early or latent viraemia phase) will not be included. This will be checked by routine blood tests and serology panels before the patient is cleared for surgery.
   Highly lipemic fluids will be discarded.
Transport: Once the biopsy procedure is planned, the participating hospital will inform PMI's laboratory coordinator for efficient sample pick up. For emergency procedures after 7 μm, samples can be stored in room temperature for next day pick up.
   Opening: The Samples received in the laboratory are opened in a vertical laminar air flow only.

### Example 2. Laboratory Method (Liver Organoid Development)

### DAY1:

1. Take 10 coverslips.
2. Prepare 25-50 μg/ml collagen solution in ddH2O.
3. Add 1 μl of collagen solution in concentric circles in 15 spots.
4. Incubate for 1 hour at 37° C.
5. Rinse slide 3 times with ddH2O.
6. Dry the coverslip thoroughly.
7. Thaw a vial of normal hepatocyte cultured in the lab.
8. Add cells at a concentration of 70000 hepatocyte in a final volume of 50 μl of DMEM incomplete medium.
9. Put the culture in the incubator.
10. Every hour, take out the slide and shake it horizontally and vertically 3-4 times.
11. Check under microscope.
12. Wash unattached cells by gentle aspiration and with DMEM (incomplete).
13. Keep in 37° C. 5% CO2 overnight.

### DAY2:

1. Thaw fibroblast cells and HUVEC cells grown in the laboratory.
2. Incubate the culture with Fibronectin 2 mg/sqcm for 37° c.
3. Mix 6000 HUVEC with 90000 fibroblasts and seed this mixture onto hepatocytes at a ratio of 1:5.
4. Incubate for 4 hours.
5. Add a layer of matrigel to the cell mix.

### DAY3:

1. Add 6000 HUVEC cells on top of the Matrigel in DMEM (complete)
2. Keep to develop organoid for 3 days
   DAY7: 1. Add chemotherapy drugs to the organoids.
   DAY8: Collect the chemodrug containing medium containing active compounds and store in −80 C.

### I] Serum and ECM Extraction:

A. Serum extraction of the patient: Blood will be drawn from the patient by standard venepuncture method in a vacutainer, transferred to a clotted vial and allowed to clot in an upright position for 30 minutes (and not more than 60 minutes). Centrifugation will be performed for 15 minutes at 2500 rpm within one hour of collection, and the supernatent serum will be aliquoted and stored at −20° C.

### B. ECM Preparation:

1. The tumour pieces will be chopped with a surgical scalpel to 1 mm³ explants.
2. Tissue slices will be suspended in dispase solution, and incubated for 15 mins at 48 C.
3. The tissues will be homogenized in a high salt buffer solution containing 0.05M Tris pH 7.4, 3.4M sodium chloride, 4 mM of EDTA, 2 mM of N-ethylmaleimide and protease and phosphatase inhibitors using tissue homogenizer.
4. The homogenized mixture will be centrifuged three times at 7,000 g for 15 min and the supernatant will be discarded to retain the pellet. The pellet will be incubated in 2M Urea buffer (0.15M sodium chloride and 0.05M Tris pH 7.4) and stirred for 1 h at 50 C.
5. The complex extracted proteins will be solubilised in Urea buffer.
6. The mixture was then finally centrifuged at 14,000 g for 20 mins and re-suspended in the 2M Urea buffer, aliquoted and stored at −80 C. This protein solution is used as extracellular matrix protein for every individual patient.

### II] Primary Tumor 3D Culture:

DAY1:
1. Serum separation will be done from the blood collected from the patient.
2. Solid tumour will be minced to 96 small pieces using scalpel blades (S2646-100EA, Sigma)
3. From stepl, tumour pieces will be put on to the wells according to the following layout:

| | | | | | | | | | | | |
|---|---|---|---|---|---|---|---|---|---|---|---|
| CON- | CON- | CON- | DRUG1 | DRUG1 | DRUG1 | DRUG1 | DRUG1 | DRUG1 | DRUG1 | DRUG1 | DRUG1 |
| TROL | TROL | TROL | DOSE1 | DOSE1 | DOSE1 | DOSE2 | DOSE2 | DOSE2 | DOSE3 | DOSE3 | DOSE3 |
| DRUG2 | DRUG2 | DRUG2 | DRUG2 | DRUG2 | DRUG2 | DRUG2 | DRUG2 | DRUG2 | DRUG3 | DRUG3 | DRUG3 |
| DOSE1 | DOSE1 | DOSE1 | DOSE2 | DOSE2 | DOSE2 | DOSE3 | DOSE3 | DOSE3 | DOSE1 | DOSE1 | DOSE1 |
| DRUG3 | DRUG3 | DRUG3 | DRUG3 | DRUG3 | DRUG3 | DRUG4 | DRUG4 | DRUG4 | DRUG4 | DRUG4 | DRUG4 |
| DOSE2 | DOSE2 | DOSE2 | DOSE3 | DOSE3 | DOSE3 | DOSE1 | DOSE1 | DOSE1 | DOSE2 | DOSE2 | DOSE2 |
| DRUG4 | DRUG4 | DRUG4 | DRUG5 | DRUG5 | DRUG5 | DRUG5 | DRUG5 | DRUG5 | DRUG5 | DRUG5 | DRUG5 |
| DOSE3 | DOSE3 | DOSE3 | DOSE1 | DOSE1 | DOSE1 | DOSE2 | DOSE2 | DOSE2 | DOSE3 | DOSE3 | DOSE3 |
| DRUG6 | DRUG6 | DRUG6 | DRUG6 | DRUG6 | DRUG6 | DRUG6 | DRUG6 | DRUG6 | DRUG7 | DRUG7 | DRUG7 |
| DOSE1 | DOSE1 | DOSE1 | DOSE2 | DOSE2 | DOSE2 | DOSE3 | DOSE3 | DOSE3 | DOSE1 | DOSE1 | DOSE1 |
| DRUG7 | DRUG7 | DRUG7 | DRUG7 | DRUG7 | DRUG7 | DRUG8 | DRUG8 | DRUG8 | DRUG8 | DRUG8 | DRUG8 |
| DOSE2 | DOSE2 | DOSE2 | DOSE3 | DOSE3 | DOSE3 | DOSE1 | DOSE1 | DOSE1 | DOSE2 | DOSE2 | DOSE2 |
| DRUG8 | DRUG8 | DRUG8 | DRUG9 | DRUG9 | DRUG9 | DRUG9 | DRUG9 | DRUG9 | DRUG9 | DRUG9 | DRUG9 |
| DOSE3 | DOSE3 | DOSE3 | DOSE1 | DOSE1 | DOSE1 | DOSE2 | DOSE2 | DOSE2 | DOSE3 | DOSE3 | DOSE3 |
| DRUG10 | DRUG10 | DRUG10 | DRUG10 | DRUG10 | DRUG10 | DRUG10 | DRUG10 | DRUG10 | CON- | CON- | CON- |
| DOSE1 | DOSE1 | DOSE1 | DOSE2 | DOSE2 | DOSE2 | DOSE3 | DOSE3 | DOSE3 | TROL | TROL | TROL |

4. 10 μl of ECM material will be layered onto the wells surrounding the tumour piece.
5. 100 μl Complete RPMI1640 (with 10% patient's serum) will be added to each well.
6. Plates will be kept in 37° C. overnight.
DAY2: Chemotherapeutic drugs will be added (after organoid treatment)
DAY3: 30 μl of Complete RPMI1640 will be added to each well.
DAY4: Chemotherapeutic drugs will be added (after organoid treatment)
DAY5: 30 μl of Complete RPMI1640 will be added to each well.
DAY6: Chemotherapeutic drugs will be added (after organoid treatment)
DAY7: 30 μl of Complete RPMI1640 will be added to each well.
DAY8: (Parallel organoids will be started to grow in the lab like the liver organoid along with the first line experiment).
   STEP1: Medium will removed from the wells treated with same drugs and mixed together. 10 μl medium will be taken to count cells. Cells will be counted using trypan blue dye exclusion method. Total medium will be divided into 4 equal parts, and administered to 4 different 3D cultured organoids.
   STEP2: 10% formalin (200 μl) will be added to 96 well plates, and the tissues will be fixed for 4 hours.
   Formalin will be discarded, and FFPE will be prepared according to standard techniques. 3 μm sections will be cut on PL slides, and stained for H&E, Ki-67, and any other special stain if needed.

### III] Generating First Line Chemo Response Report:

All the sections will be evaluated for Chemo-induced necrosis and will be scored according to percent of cell necrosis.
Report will include:
1. Cell detachment function
2. Apoptosis percentage
3. Necrosis percentage
4. Best chemo option
5. 1st line chemotherapy resistance

### IV] Follow Up Patients Every 3 Months for Tumor Size, Morbidity Etc:

1. CT scan (with contrast) every 3 months
2. CBC, LFT, RFT
3. Charlson Co-morbidity index (CCI)

### V] Collection of Non-Adhering Chemo-Resistant Surviving Cells:

Using a pipette, 200 microlitre of medium containing non-adherent cells will be pooled from the same drug-treated wells as same treatment group irrespective of the dosing of chemotherapy given. Wells will be washed with PBS twice and pooled in the same drug treated group. Cells will be counted in a Neubauer hemocytometer using trypan blue dye exclusion method.
DAY8 Continued: Non-adhering chemo-resistant surviving cells will be given to different organoids in culture. The cultures will be maintained till day 23.

### DAY23:

Chemotherapeutic drugs will be added (after liver organoid treatment) (1st).

### DAY25:

30 μl of Complete DMEM/RPMI1640 will be added to each well.

### DAY27:

Chemotherapeutic drugs will be added (after liver organoid treatment) (2nd)

### DAY29:

30 μl of Complete DMEM/RPMI1640 will be added to each well.

### DAY31:

Chemotherapeutic drugs will be added (after liver organoid treatment)(3rd)

### DAY33:

30 μl of Complete DMEM/RPMI1640 will be added to each well.

### DAY34:

10% formalin (200 μl) will be added, and the tissues will be fixed for 4 hours. Formalin will be discarded, and FFPE will be prepared according to standard techniques. 3 μm sections will be cut on PL slides, and stained for H&E, Ki-67, and any other special stain if needed.
Report will include:
Apoptosis percentage
Necrosis percentage
Best chemo option

### VI] Generation of Second Line Chemo Report.

10% formalin (200 μl) will be added, and the tissues will be fixed for 4 hours. Formalin will be discarded, and FFPE will be prepared according to standard techniques. 3 μm sections will be cut on PL slides, and stained for H&E, Ki-67, and any other special stain if needed.
Report will include:
Apoptosis percentage
Necrosis percentage
Best 2^{nd} line chemo option
   VII] Follow Up of Patients at Every 3 Month Interval for 2 Year: Follow up will be done with CT scans (contrast enhanced), CBC, LFT, RFT, and Charlson Co-morbidity index (CCI) every 3 months for 2 years after the initiation of chemotherapy.
   VIII] Correlation Curves with Laboratory Generated Data and Patient Data Will be Accrued to Generate Statistical Data for Predicting Time to Recurrence and Organ of Metastasis.

The entire screening procedure may require 2-3 weeks to complete, depending upon the cell growth demonstrated after the first-line chemotherapy. However, at the end of that period, the clinician has already identified the most appropriate second-line chemotherapeutic agent to use for a particular patient, should the cancer recur in that patient.

In one embodiment of the invention, the presently disclosed screening procedure may include a method of screening chemotherapeutics for second-line chemotherapy, where the method comprises:
culturing cancer cells of interest to prepare plural first cultures;
exposing each of the first cultures to one of a plurality of first chemotherapeutic agents;
identifying a most effective member of the plurality of first chemotherapeutic agents;
culturing cancer cells that survived exposure to the most effective first chemotherapeutic agent to prepare plural second cultures;
exposing each of the second cultures of cancer cells to one of a plurality of second chemotherapeutic agents; and
identifying a most effective member of the plurality of second chemotherapeutic agents.

Each of the first and second cultures of the method may be prepared on a substrate.

The substrate may include a biological polymer.

The substrate may include a proteinaceous matrix.

The substrate may include a monolayer of normal cells.

The normal cells and cancer cells of interest may be collected from a single patient.

The plurality of first and/or second chemotherapeutic agents may include one or more of paclitaxel, carboplatin, cisplatin, adriamycin, gemcitabine, topotecan, etoposide, docataxel, ifosamide, and 5-fluoro uracil.

The method of screening may be performed using a multiwall microplate.

In another embodiment of the invention, the presently disclosed screening procedure may include a method comprising:
preparing plural substrates, each substrate including a layer of normal cells on an organic matrix;
culturing cancer cells of interest on the prepared substrates to prepare plural first cultures;
exposing each of the first cultures to one of a plurality of first chemotherapeutic agents;

The presently disclosed assay provides significant advantages over currently available chemopredictive assays. In particular, where an appropriate substrate is used, the disclosed chemopredictive assay provides an authentic ex vivo environment, such as where the substrate includes an extracellular matrix and/or the use of normal cells obtained from the patient of interest in the region where the tumor exists.

The present chemopredictive assay includes a screening process for chemotherapeutic agents, where the screening process determines the effectiveness of the chemotherapeutic agents against recurring cancer cells. As set out in FIG. 1, a representative example of a method of the present invention is depicted in flowchart 10, and includes culturing cancer cells of interest to prepare plural first cultures at 12, exposing each of the first cultures to one of a plurality of first chemotherapeutic agents at 14, identifying a most effective member of the plurality of first chemotherapeutic agents at 16, culturing cancer cells that survived exposure to the most effective first chemotherapeutic agent to prepare plural second cultures at 18, exposing each of the second cultures of cancer cells to one of a plurality of second chemotherapeutic agents at 20, and identifying a most effective member of the plurality of second chemotherapeutic agents at 22.

In general, the present assay is performed under conditions selected to mimic the environment in which the cancer cells of interest exist, optionally including an extracellular matrix and/or a monolayer of normal cells upon a selected substrate. In this environment, selected tumor cells are challenged with multiple chemotherapeutic candidate drugs and a first-line selection of chemotherapy agent is performed, for example by counting the cancer cells that remain attached to an extracellular matrix and monolayer of normal cells.

Subsequent second-line chemotherapeutic selection is performed by assessing the ability of cells that were exposed to the first-line chemotherapeutic agent to grow into secondary colonies, and their ability to remain attached to the substrate after exposure to a second-line chemotherapeutic agent. The second-line selection of chemotherapeutic agent is based upon the ability of the surviving cancer cells to remain viable after exposure to a variety of second-line chemotherapeutic agents.

Substrate. Cell colonies, either of normal cells or of cancer cells, are typically prepared upon some type of supporting substrate. The substrate may be as basic as the surface of a microwell plate. However, the predictive value of the present screening method may be enhanced by preparing a substrate that more closely resembles the environment within the patient.

In one aspect, the substrate includes a matrix, typically an organic matrix. The matrix may be composed of one or more biological polymers. The matrix may include proteins, and may be a solid or semi-solid matrix. In one embodiment, the matrix includes MATRIGEL, a gelatinous mixture of proteins (BD Biosciences).

The substrate may be further enhanced by preparing an environment of normal cells collected from the vicinity of the collected cancer cells. For example, normal cells may be cultured in order to prepare a substrate that includes at least a monolayer of normal cells.

The chemotherapeutic agents under evaluation in the present screening process may include any agent of interest selected by the physician. Typically the chemotherapeutic agent will be a drug that has been recognized as having efficacy in chemotherapy. In one embodiment of the invention, the chemotherapeutic agents being screened includes one or more of paclitaxel, carboplatin, cisplatin, adriamycin, gemcitabine, topotecan, etoposide, docataxel, ifosamide, and 5-fluoro uracil.

### EXAMPLES

Example 1. Procurement of Tissue: The Cancer Tissue of Interest is Procured During cancer surgery from the operating room under sterile conditions. Typically, the performing surgeon removes a piece of the tumor and transfers it into a sterile 50 ml tube containing 10 ml of sterile RPMI1640 medium (without Fetal Bovine Serum or FBS). The surgeon then uses a cervical brush to collect normal peritoneal cells from the organ of choice of the surgeon, and the brush is transferred to a 15 ml sterile tube containing 5 ml of RPMI1640 (without FBS). The two tubes are then transferred to the laboratory under room temperature conditions in a sterile box. Using this method, the sample can remain stable up to three days post-surgery.

Example 2. Laboratory method: The normal cells collected using the cervical brush are harvested under sterile conditions using 10 ml RPMI1640 medium (with 10% FBS). The cells are counted and 100 μl of the cell suspension are added to a matrigel (Becton Dickinson) coated 96-well microplate and incubated in a CO₂ incubator under 5% CO₂ and 37° C. for 2 hours. The cancer tumor is transferred under sterile conditions on a 60 mm dish and a pure tumor piece (i.e., without surrounding tissues) having a size of 2-4 mm is surgically excised. The tumor is injected 50 times with 50 ml of RPMI160 medium (with 10% FBS) using a 10 ml syringe fitted with a 26 gauge needle. The effused cell suspension is collected in a 50 ml tube, the cells are washed twice with PBS (phosphate buffered saline) and then re-suspended in 10 ml RPMI160 medium (with 10% FBS). The cancer cells are counted.

After the normal cells are incubated two hours, and after microscopic observation confirms that the normal cells have adhered and formed a monolayer on the plate, 100 μl of tumor cells are added on top of the normal cells. The plates are kept overnight in 5% CO₂ and 37° C. in a CO₂ incubator. After 18 additional hours, RPMI1640 medium is removed and 100 μl fresh medium is added. In the 96-well microplate, row A1-A12 is used as Control (without drug) and in rows B to H, seven different chemotherapeutic agents are added as per the following protocol, in triplicates. The individual chemotherapeutic agents are chosen according the physicians' requirements for the particular type of cancer involved, and they are used at a dose that is within the AUC for each particular drug.
B1-B3=Drug 1 (dose 1)
B4-B6=Drug 1 (dose 2)
B7-B9=Drug 1 (dose3)
B10-B12=Drug 1 (dose 4)

After drug addition, the microplates are incubated in 5% CO₂ and 37° C. in a CO₂ incubator. The next day a second round of chemotherapeutic agents are applied according to the same protocol used initially.

After an additional day, or 48 hours after the initial drug treatment, the medium from the wells corresponding to the same chemotherapeutic agent are collected in a 15 ml tube (i.e., the medium from B1 to B12 is collected in the same tube). The microplate wells are washed twice with PBS and the washings are collected in the same tube. The resulting suspensions include floating cells that have responded to the chemotherapeutic agents and have either died or floated in the medium. The plates are then fixed for 15 minutes in 100% methanol and stained with Cell stain solution (Chemicon, CA) for 5 minutes. The stain is then washed away.

The best functional drug for first-line chemotherapy is identified by calculating the following ratio:

$$\frac{[ \begin{matrix}( number of normal cells added ) - \\ ( number of tumor cells added )\end{matrix} ]}{[ \begin{matrix}( number of remaining normal cells ) - \\ ( number of remaining tumor cells )\end{matrix} ]}$$

This calculation takes into account the toxicity of the chemotherapeutic agent to normal cells, as well as the toxicity toward tumor cells. The calculation is performed using an automated inverted microscope (Olympus IX81 with motorized stage) followed by image analysis with Imagepro software, and the resulting value is immediately reported to the clinician.

Once the best functional first-line chemotherapeutic agent is identified, the tube of cells treated with the agent is washed with PBS and the viable cells are counted. The cells are then re-suspended in 10 ml RPMI1640 medium (with 10% FBS) and an equal number of cells are added to a 96-well nanoculture microhoneycomb plate (SCIVAX) and incubated in 5% CO₂ and 37° C. in a CO₂ incubator. After 5-10 days of incubation, colonies of cells derived from the chemotherapeutic-challenged cells being to appear. These cells are allowed to grow to about 50% confluence.

The resulting cell colonies are then again subjected to an array of chemotherapeutic agents, and the screening process is carried out as described above. The best functional drug for second-line chemotherapy is then identified by calculating

(number of tumor cells added)−(number of tumor cells remaining in the plate)

where a greater numerical value predicts that the corresponding chemotherapeutic agent exhibits greater efficacy for a patient who has already undergone first-line chemotherapy, and in whom the disease has recurred. This prediction is immediately reported to the clinician.

The entire screening procedure may require 2-3 weeks to complete, depending upon the cell growth demonstrated after the first-line chemotherapy. However, at the end of that period, the clinician has already identified the most appropriate second-line chemotherapeutic agent to use for a particular patient, should the cancer recur in that patient.

In one embodiment of the invention, the presently disclosed screening procedure may include a method of screening chemotherapeutics for second-line chemotherapy, where the method comprises:
culturing cancer cells of interest to prepare plural first cultures;
exposing each of the first cultures to one of a plurality of first chemotherapeutic agents;
identifying a most effective member of the plurality of first chemotherapeutic agents;
culturing cancer cells that survived exposure to the most effective first chemotherapeutic agent to prepare plural second cultures;
exposing each of the second cultures of cancer cells to one of a plurality of second chemotherapeutic agents; and
identifying a most effective member of the plurality of second chemotherapeutic agents.

Each of the first and second cultures of the method may be prepared on a substrate.

The substrate may include a biological polymer.

The substrate may include a proteinaceous matrix.

The substrate may include a monolayer of normal cells.

The normal cells and cancer cells of interest may be collected from a single patient.

The plurality of first and/or second chemotherapeutic agents may include one or more of paclitaxel, carboplatin, cisplatin, adriamycin, gemcitabine, topotecan, etoposide, docataxel, ifosamide, and 5-fluoro uracil.

The method of screening may be performed using a multiwall microplate.

In another embodiment of the invention, the presently disclosed screening procedure may include a method comprising:
preparing plural substrates, each substrate including a layer of normal cells on an organic matrix;
culturing cancer cells of interest on the prepared substrates to prepare plural first cultures;
exposing each of the first cultures to one of a plurality of first chemotherapeutic agents;
identifying a most effective member of the plurality of first chemotherapeutic agents using a formula

$$\frac{[ \begin{matrix}( number of normal cells added ) - \\ ( number of tumor cells added )\end{matrix} ]}{[ \begin{matrix}( number of remaining normal cells ) - \\ ( number of remaining tumor cells )\end{matrix} ]}$$

culturing cancer cells that survived exposure to the most effective first chemotherapeutic agent on the prepared substrates to prepare plural second cultures;
exposing each of the second cultures of cancer cells to one of a plurality of second chemotherapeutic agents; and
identifying a most effective member of the plurality of second chemotherapeutic agents using a formula

(number of tumor cells added)−(number of tumor cells remaining).

As noted above, the present inventions also pertain to cancer and ovarian cancer treatment, and particularly, to inventions that identify biomarkers to predict chemo-resistance in cancer and ovarian cancer. Further, the inventions relate to identification of: (i) biomarkers for predicting up-front carboplatin paclitaxel resistance and (ii) targets that can reverse chemo-resistance in ovarian cancer using an in silico approach.
1. Carboplatin and paclitaxel related genes over-expressed in clear cell carcinoma in comparison to serous carcinoma. Can data predict up-front resistance, delayed resistance and sensitive patient?

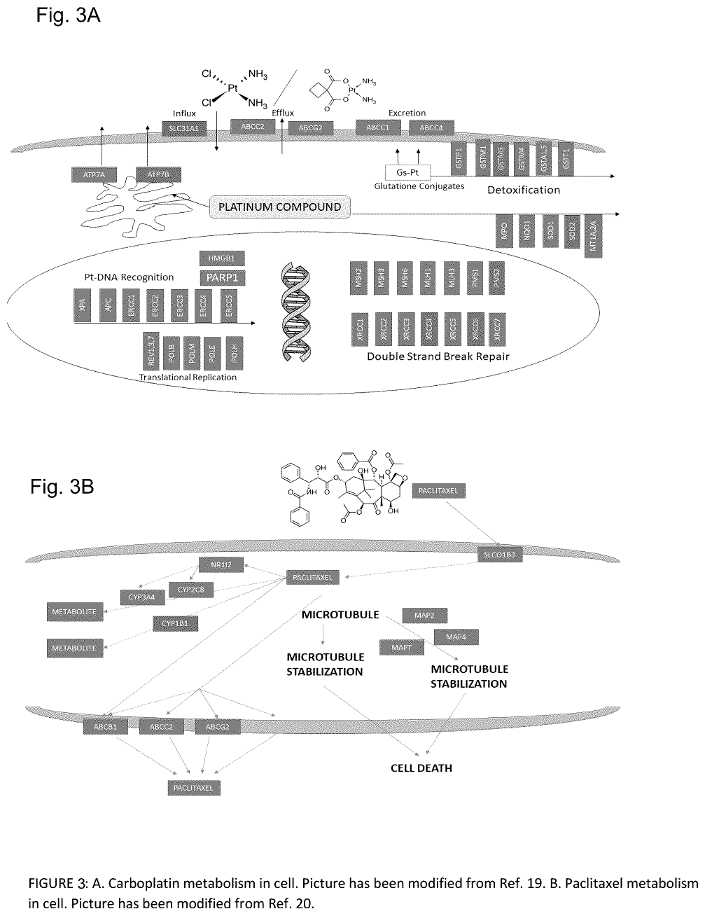

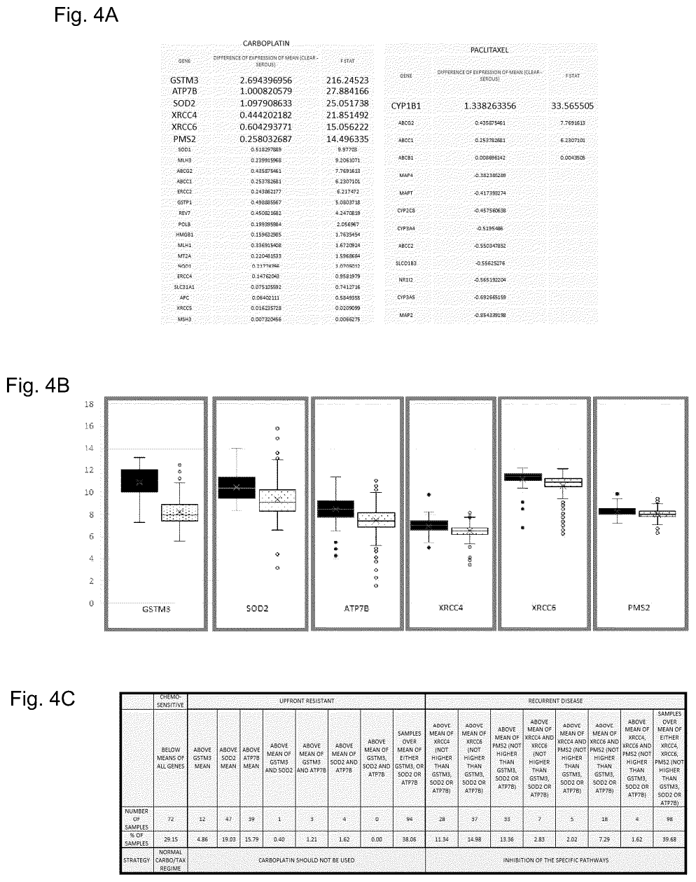

Referring to FIG. 3, the expression profiles of genes related to both carboplatin and paclitaxel were downloaded. Data for the genes REV3 and MT1 A were not available on the GENT2 website. Mean of expression of genes in serous carcinoma were subtracted from the mean of expression of the same genes in clear cell carcinoma. A positive value indicated that overall expression of the genes was higher in clear cell carcinoma when compared to serous and a negative value indicated that the expression levels in serous carcinoma were higher. Statistical analysis revealed that GSTM3, ATP7B and SOD2 were significantly higher in clear cell carcinoma (FIG. 4A). The genes with statistically significant difference with over-expression in clear cell carcinoma are shown in FIG. 4A in bold and bigger fonts. GSTM3 and SOD2 are responsible for the detoxification of carboplatin while ATP7B is responsible for efflux of carboplatin. Higher expression of these three genes in clear cell carcinoma can explain the failure to carboplatin treatment (FIG. 4A). Post carboplatin induced double stranded break of DNA (DSB), XRCC4, XRCC6 and PMS2 will be involved in repairing the DNA through the non-homologous end joining (NHEJ) (23,46). These three genes are statistically significant in difference and are up-regulated in clear cell compared to serous carcinoma. Role of these three genes can be implicated in generating gradual resistance to carboplatin (FIG. 4A). CYP1B1 is responsible for metabolism of Paclitaxel and this is the only gene that is up-regulated in clear cell compared to serous carcinoma of the ovary and is statistically significant (FIG. 4A). As shown in a box and whisker chart in FIG. 4B, all of these genes are up-regulated in clear cell carcinoma, are statistically significant and can define both up-front and gradual chemo-resistance to carboplatin and paclitaxel.

The individual values of serous ovarian carcinoma gene expression were subsequently subtracted from the mean of individual genes of clear cell carcinoma. The hypothesis was that values of serous carcinoma above the mean of clear cell carcinoma will define chemo-resistance by the respective genes. The entire calculation was done in excel and is given in Supplement 2. As shown in FIG. 4C, 29.15% of samples had values of all the genes related to carboplatin less than clear cell mean. We label these samples as responders to first line carboplatin therapy. 38.06% of samples had higher values of GSTM3 or ATP7B or SOD2 either singularly or in combination implicating that the probability of these samples showing resistance to carboplatin was very high. By our hypothesis, these samples can be labeled as up-front resistant. 39.68% of serous samples had values over mean of clear cell carcinoma of XRCC4 or XRCC6 or PMS2 either singularly or in combination and these samples demonstrated GSTM3, ATP7B or SOD2 lower than the mean of clear cell carcinoma (FIG. 4C). We label these samples as responders but would show carboplatin resistance eventually leading to recurrent disease.
2. Plasma membrane genes up-regulated in clear cell carcinoma in comparison to serous carcinoma.

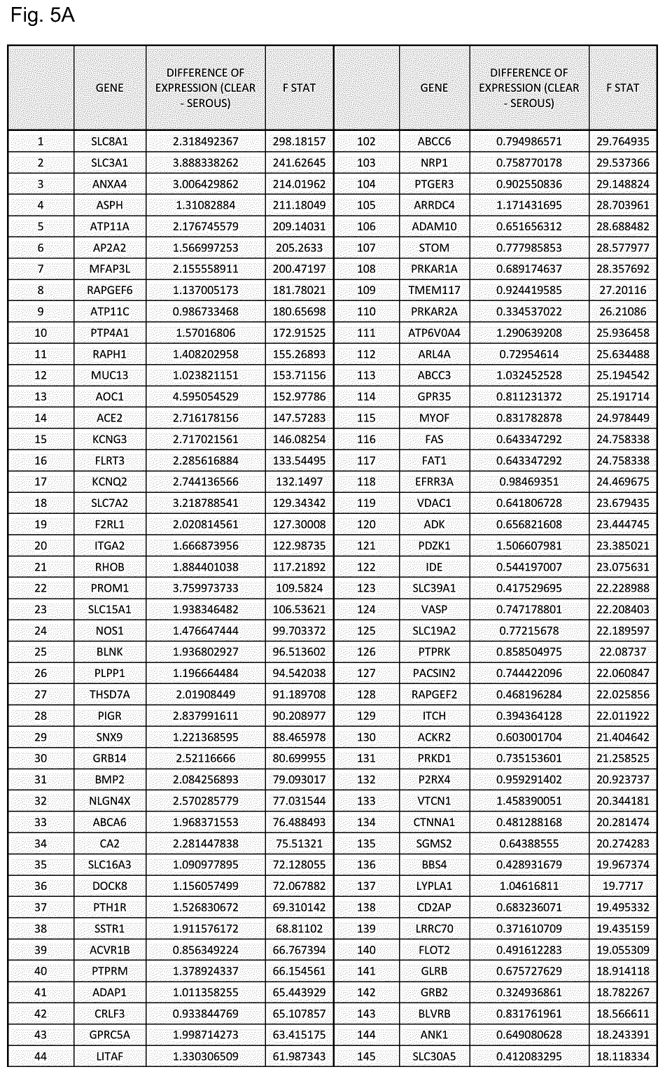

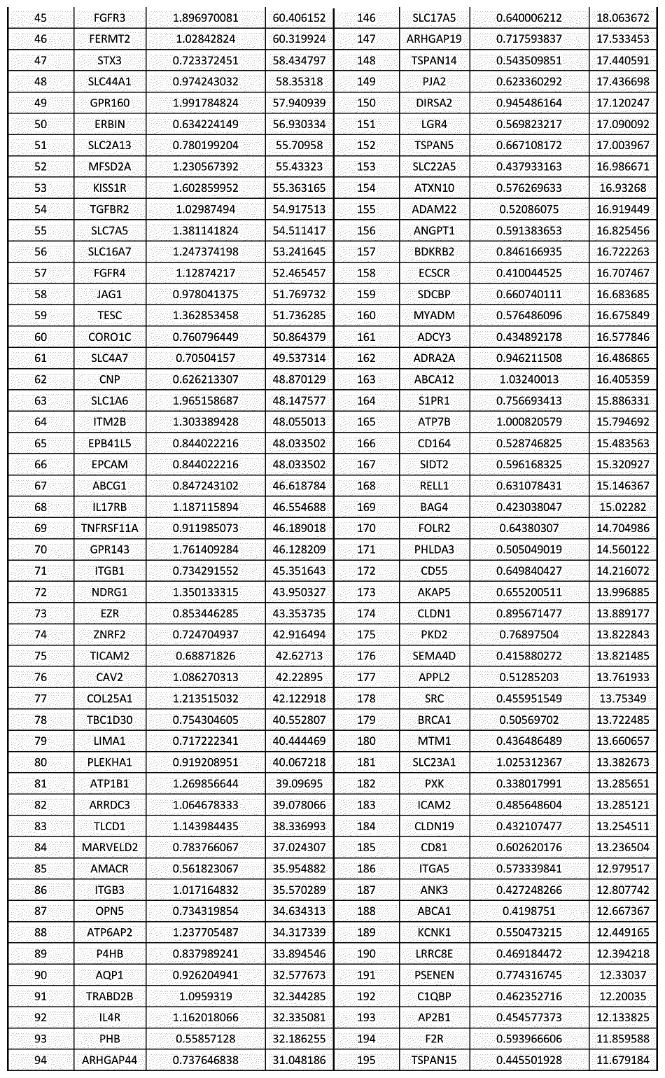

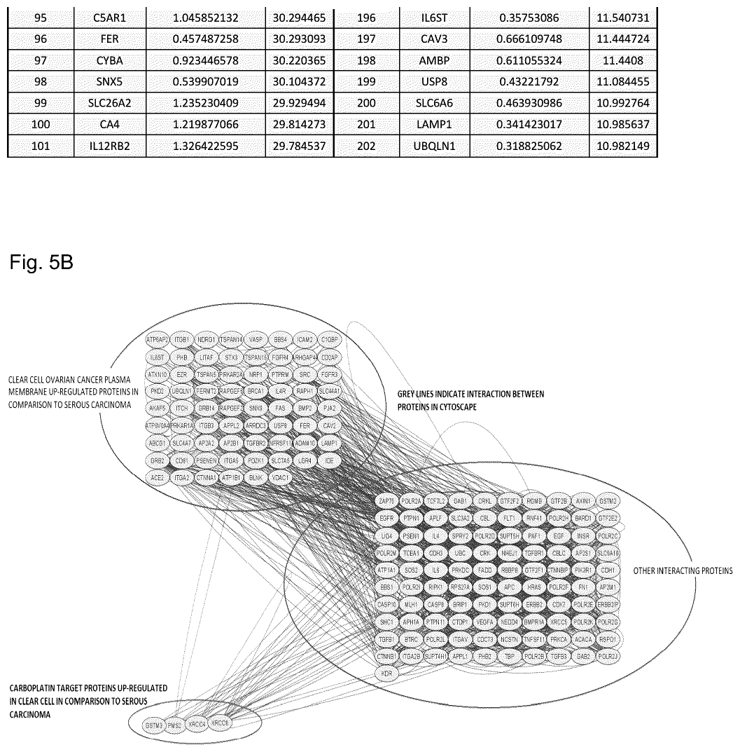

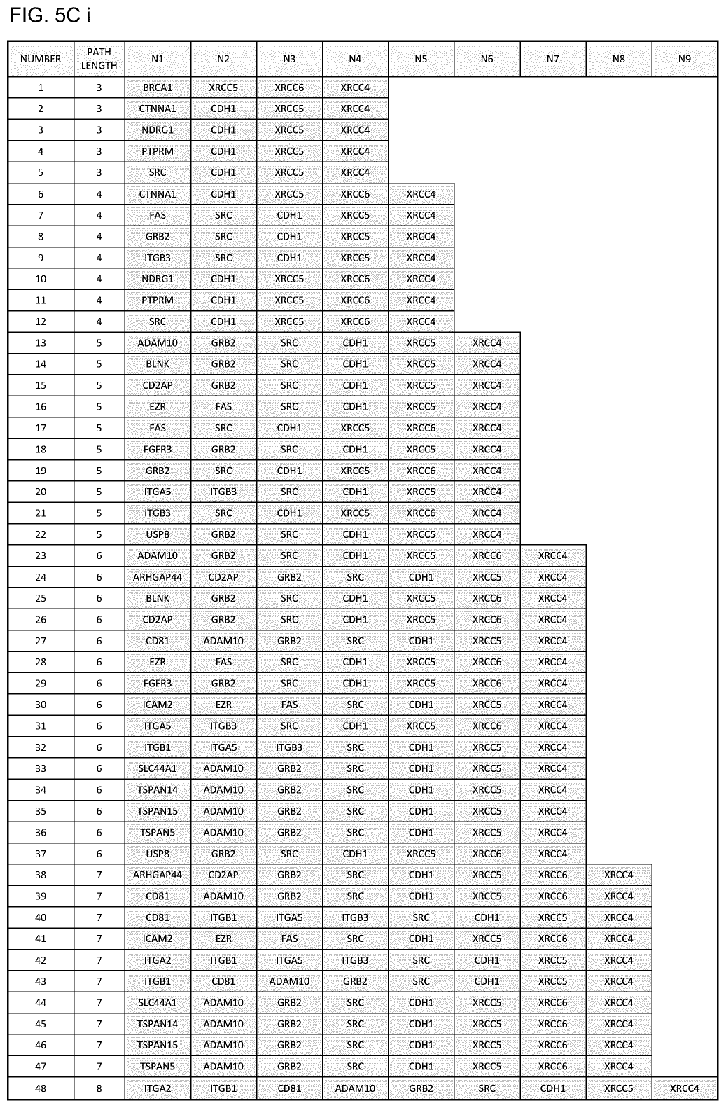

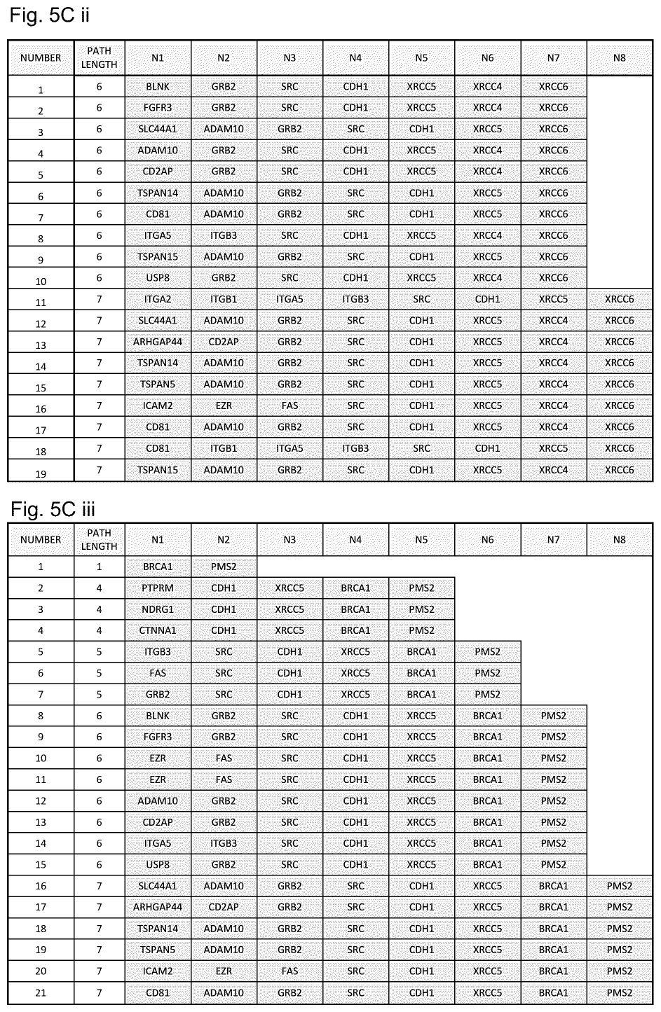

As demonstrated in the methods, we listed plasma membrane proteins in Homo sapiens (24) and downloaded the gene expression values from the GENT website (25). A total of 1522 plasma membrane protein gene expression data were downloaded. First, the mean of expression of the individual genes were calculated for both clear cell and serous carcinoma of the ovary. 544 genes had mean expression values higher in clear cell compared to serous carcinoma. These expression datasets were statistically evaluated and 202 genes demonstrated significant difference between clear cell and serous carcinoma (FIG. 5A).
3. Construction of protein-protein interaction network from plasma membrane proteins to carboplatin and paclitaxel related genes followed by identification of pathways leading to activation of carboplatin and paclitaxel related genes.

We listed the plasma membrane proteins that were up-regulated in clear cell carcinoma compared to serous carcinoma that were statistically significant. We also listed carboplatin and paclitaxel drug related proteins that were up-regulated in clear cell carcinoma and were statistically significant. These proteins are marked in red in FIGS. 3A and 3B. Using https://string-db.org/ (26) as mentioned in the methods section, we created a network and visualized the network in Cytoscape 3.8.2 (27) (FIG. 5B). We have listed the interconnecting protein nodes that were generated because of the network and downloaded their expression data from the GENT2 website. Again the up-regulated and statistically significant genes in clear cell carcinoma in comparison to serous were listed. Using Pathlinker plugin in Cytoscape 3.8.2 we had extracted the pathways from the source nodes of plasma membrane to the drug related proteins. After filtering for only the up-regulated and statistically significant genes in clear cell carcinoma compared to serous in excel, we identified the pathways where all the genes were up-regulated and statistically significant in clear cell carcinoma (FIG. 5C). The network did not include GSTM3, SOD2, ATP7B, CYP1 B1 and XRCC5 as no pathways with experimental validation could be determined. However, we have included XRCC5 in the pathways as the role of XRCC6 is dependent on the binding of XRCC5.
4. Identification of off-label drugs that can target pathways to inhibit chemo-resistant pathways.

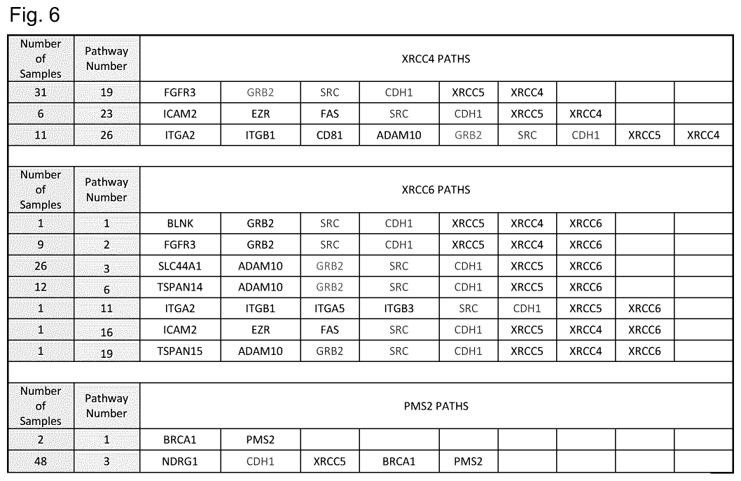

To use these pathways to determine which pathways can have the best potential to be knocked down, we used our mathematical algorithm to identify the pathway strengths. FIG. 6 shows the pathways that are maximally activated for stimulating XRCC4, XRCC6 and PMS2 function that can lead to DNA repair post Carboplatin treatment. CDH1 is relevant in all the pathways except for one pathway where BRCA1 can directly activate PMS2. The other most relevant molecule is SRC that is also involved in all the pathways for XRCC4 and XRCC6 activation. GRB2 is the third most relevant molecule that is involved in majority of the pathways leading to XRCC4 and XRCC6 activation. FGFR3, ICAM2 and ITGA2 are the plasma membrane proteins that can serve as a start point of the pathways leading to XRCC4 activation. BLNK, FGFR3, SLC44 A1, TSPAN 14, TSPAN 15, ITGA2 and ICAM2 are the start points of the pathways leading to XRCC6 activation. BRCA1 and NDRG1 are the start points for PSM2 activation (FIG. 6).

While there are no commercially available drugs for CDH1, SRC kinase inhibitors are widely used in the management of chronic myeloid leukemia (CML) patients. Drugs like Dasatinib and Saracatinib inhibit SRC leading to inhibition of SRC related pathways. Our results therefore indicate a potential role of SRC kinase inhibitors in reversing chemo-resistance to Carboplatin.
1. IDENTIFICATION OF PATIENTS UP-FRONT RESISTANT TO CARBOPLATIN BY TESTING FOR GSTM3, ATP7B, SOD2 EXPRESSION AND PACLITAXEL BY CYP1 B1 EXPRESSION.

As shown in FIG. 3A, Carboplatin is neutralized in the cell by the Glutathione pathway where GSTM3 is an important component. In several cancers, members of the glutathione S-transferase (GST) family have been reported as being overexpressed and in most cases have been linked to poor prognosis and chemo-resistance (28-31). ATP7B has been shown to over-expressed in cisplatin resistant ovarian cancer cell lines. Attenuation of ATP7B by siRNA approaches have shown to restore chemo-sensitivity in these cell lines (32). Mitochondrial superoxide dismutase (SOD2) is an enzyme that metabolizes superoxide in mitochondria and plays an important role in maintaining mitochondrial function through oxidative stress tolerance. SOD2 overexpression correlates with poor prognosis in several cancers (33-35). Suppression of SOD2 enhances ROS production in ovarian cancer cells and results in increased apoptosis, inhibition of proliferation, and enhanced sensitivity to chemotherapy (36). Our results indicated that GSTM3, SOD2 and ATP7B are up-regulated in clear cell carcinoma when compared to serous carcinoma of the ovary. This can explain the lowered chemo-sensitivity of carboplatin in clear cell carcinoma than serous carcinoma. Quantifying GSTM3, SOD2 and ATP7B by standard QPCR or RNA Seq approaches and running a comparison study between responders and non-responders to carboplatin treatment in ovarian cancer patients can open up a new diagnostic algorithm to identify non-responders to the drug.

Paclitaxel resistance is generally a lessor concern for clinicians treating ovarian cancer patients. However, it has been demonstrated in a mouse model that knocking down CYP1 B1 reduces resistance to paclitaxel (37). Our results indicate that CYP1 B1 is expressed more in clear cell carcinoma in comparison to serous carcinomas, suggesting that paclitaxel resistance can be conferred in ovarian cancer patients with CYP1 B1.

While there are no available drugs in clinical practice to down-regulate expression levels of GSTM3, SOD2, ATP7B that can be used to reverse carboplatin resistance, or drugs against CYP1 B1 to reverse paclitaxel resistance, quantification of these genes can lead to identification of up-front resistant patients. This will reduce unnecessary use of carboplatin paclitaxel chemotherapy in ovarian cancer patients who will be not respond to the drugs anyway. Specially in a neo-adjuvant setting of ovarian cancer management, the clinicians can use other chemotherapeutic regimes instead of subjecting the patients to standard therapy expecting better outcomes.
2. IDENTIFICATION OF PATIENTS WITH POSSIBILITY TO RECUR ON CARBOPLATIN THERAPY WITH PMS2, XRCC4 AND XRCC5 OVEREXPRESSION.

Carboplatin-resistant ovarian cancer cells showed the high levels of γH2AX foci formed at the basal level, and the levels of γH2AX foci remained even after the recovery time, suggesting that the DNA damage response and repair machinery were severely attenuated by carboplatin-resistance. Surprisingly, the expression levels of XRCC4, a critical factor in non-homologous end joining (NHEJ) DNA repair, were significantly decreased in carboplatin-resistant SKOV3 compared with those in non-resistant controls. Furthermore, restoration of NHEJ in carboplatin-resistant SKOV3 by suppression of ABCB1 and/or AR re-sensitizes carboplatin-resistant cells to genotoxic stress and reduces their proliferation ability. Therefore, attenuation of the NHEJ DNA repair machinery mediated by resistance to genotoxic stress might be a critical cause of chemoresistance in patients with ovarian cancer. XRCC6 over—expression has also been shown to promote resistance to cisplatin in HNSCC cell lines (38). It has also been shown that use of cisplatin up-regulates XRCC6 expression (38). Damia et al extensively reviewed the roles of PMS2 in conferring platinum resistance in ovarian cancer by working on the nucleotide excision repair (NER) and the mismatch repair (MMR) pathways (39,44). Our results indicate that XRCC4, XRCC6 and PMS2 are significantly up-regulated in clear cell carcinoma when compared to serous carcinoma of the ovary. This explains the failure of platinum drugs to work in a clear cell carcinoma setting. Higher expression of these three genes can give us an idea of which patients will recur. An objective study correlating expression levels of XRCC4, XRCC6, and PMS2 along with time to recurrence (TTR) in ovarian cancer patients can help us to improve end-point management. 3. IDENTIFICATION OF PATHWAYS LEADING TO RESISTANCE ON CARBOPLATIN THERAPY AND IF DASATINIB CAN A DRUG LEADING TO OVERCOMING OF RESISTANCE.

Using our mathematical algorithm of determining the strongest path based on expression values and out-degrees of a protein or node, and using the PFSC plugin in cytoscape (27), we have identified pathways in individual samples that can stimulate XRCC4, XRCC6 and PMS2 for correcting double stranded DNA breaks as shown in FIG. 4. In all the pathways originating from the plasma membrane and leading to XRCC4, XRCC6 and PMS2, the most common molecule that is involved is CDH1. This is highlighted in red in FIG. 4. The second most common molecule is SRC (also indicated in red in FIG. 4). While there are no commercially available clinical drugs for inhibiting CDH1, SRC inhibition is commonly used in clinical practice of chronic myeloid leukemia (CML) by using Dasatinib or Saracatinib. Saracatinib does not improve clinical efficiency of paclitaxel in carboplatin resistant ovarian cancers (40). This is obvious, as inhibition of SRC can reverse carboplatin response. Similarly, Dasatinib as a single agent therapy did not improve response of patients in recurrent ovarian cancer (41). However, it was shown that Dasatinib is safe to use as a drug with good tolerability (41). Synergistic use of Dasatinib in combination with standard chemotherapeutic agents appears to interact in a synergistic manner in some ovarian cancer cell lines (42). Combined treatment with dasatinib and paclitaxel markedly inhibited proliferation and promoted apoptosis of ovarian cancer cells, compared with control cells. Combined dasatinib and paclitaxel treatment exhibited antitumor activities in vivo and in vitro (combination indices, 0.25-0.93 and 0.31-0.75; and tumor growth inhibitory rates, 76.7% and 58.5%, in A2780 and H08910 cell lines, respectively), compared with paclitaxel treatment alone (43). While there is potential for SRC inhibition to manage ovarian cancer, subsequent trials with SRC inhibitors followed by carboplatin treatment in carboplatin failure patients can be explored. This can change the management paradigm of carboplatin treatment failure patients.

The above-described inventions have demonstrated that use of GSTM3, ATP7B and SOD2 has the potential to become a diagnostic method to detect upfront resistance to carboplatin and use of CYP1 B1 to detect upfront resistance of paclitaxel in ovarian cancer patients. Use of XRCC4, XRCC6 and PMS2 can identify ovarian cancer patients who have the potential to recur on carboplatin treatment. Our work also demonstrated the possibility of using SRC inhibitors with carboplatin in carboplatin failure patients. Use of these molecular testing can change the entire dynamics of ovarian cancer management.

### Methods Used:

1. RNA Seq data: Gene Expression database of Normal and Tumor tissues 2 (GENT2) is an updated version of GENT, which has provided a user-friendly search platform for gene expression patterns across different normal and tumor tissues compiled from public gene expression data sets. (25). GENT2 provides gene expression across 72 different tissue types and provides an option to study the differential expression and its prognostic significance based on tumor subtypes (25). Using the subtype profile tab on the webpage, Ovary was selected as the tissue type, subtype was chosen as histology, prognosis was chosen as OS (overall survival) and the gene name was given in Gene Symbol. Data was then downloaded containing RNA expression data. From the data, samples of the clear cell subtype and the serous subtype were identified and separated.
2. Statistical Analysis: A one-way model I ANOVA was done here to distinguish if there is any significant difference between the RNA seq. data of clear cell carcinoma and serous carcinoma. We calculated the F values and compared it with critical F values to find out the difference of the scores. Using the F distribution table for α=0.001 and df=(1, ∞). The F value was 10.828 which was considered as cut off value.
3. Identification of plasma membrane proteins: Plasma membrane proteins in Homo sapiens were identified through: http://amigo.geneontology.org/amigo/term/GO:0005886 (24). The plasma membrane proteins of Homo sapiens were listed in excel and were manually checked through www.uniprot.org website's subcellular location. Both the UniProt Annotation and GO-Cellular Component were checked and the publications were manually checked for curation of the plasma membrane list. The list of plasma membrane proteins is given as Supplement 2. It was important to check manually as proteins have different locations leading to different functions. Many of the proteins had different sublocations along with the plasma membrane. They were not excluded from the list.
4. Construction of interactome network: To create a drug resistance network of proteins, the drug related proteins and the plasma membrane proteins that showed higher expression in clear cell ovarian carcinoma compared to serous carcinoma and showed statistical significance were plugged into https://string-db.org/ (26) and using multiple protein tab, the organism was selected as Homo sapiens. In the settings tab, “Full String Network” was selected along with “Evidence”, and in the active interaction sources, only “Experiments” were selected. Minimum interaction score was set at 0.4 and maximum number of interactors were set at 50. The data was downloaded as a tabular file and it was opened in Cytoscape 3.8.2 software (27). The pathways were extracted using Pathlinker plugin and the centralities were calculated using the Centiscape plugin. Each of the interactions (edges) were cross checked manually to determine if the interaction is experimentally shown in any human cell type. The main idea was if two proteins have been shown to be interacting in a human cell line, then the possibility of them interacting in ovarian cancer is highly elevated. Previous work has shown that even if the proteins are up-regulated in a cancer type, they may not work through a defined pathway in all the cell types. For example, AKT1 has been shown to be activated by 18:1 Lysophosphatidic acid (LPA), using Mitogen Activated Protein Kinase Kinase and p38 Mitogen Activated Protein Kinase in ovarian cancer cell lines but not in breast cancer cell lines (34,45). However, we assumed that over-expression of a particular protein can be a determinant for sending signals for drug management in the cell. Once the connecting proteins were determined, they were evaluated for over-expression in clear cell carcinoma compared to serous and the statistically significant molecules were taken to determine the resistant pathways. The only exception was XRCC5, as XRCC5 was not statistically significant between the clear cell and the serous groups, but XRCC5 binds to XRCC6 to provide a functional entity for XRCC4 to work.
5. Pathway strength calculation: While trying to calculate for pathway strength, we identified every individual protein as Node (N). We rationalized that to calculate pathway strength, the Node strength (strength of the source protein contributing or interacting with the target protein defined as NS) need to be identified. Therefore, NS will vary according to the expression of the source node (E). NS will vary inversely to the number of output paths (O). In biological terms this means the probability of a node or protein to send signals to different target proteins. This can be written as

### NS≢E and

$$NS ∝ \frac{1}{O} NS ∝ \frac{E}{O} = k . \frac{E}{O}$$

Where k is the constant.
Path length is denoted as PL and Path Strength (PS) can be calculated as the sum of Node strengths divided by path length.
Path Strength can be then denoted as

$$\sum \frac{NS}{PL}$$

We have also calculated pathway strengths using PSFC, a pathway signal flow calculator plugin in cytoscape (31) with rule F, where

$$Activation ( + ) = source + target$$

$$( expression of source and expression of target )$$

$$Inhibition ( - ) = source - target$$

$$( expression of source and expression of target )$$

$$Multiple input signals = Add ( Number of input signals )$$

$$Splitting = Equal , Out ( Number of out degrees )$$

The presently disclosed assay provides significant advantages over currently available chemopredictive assays. In particular, where an appropriate substrate is used, the disclosed chemopredictive assay provides an authentic ex vivo environment, such as where the substrate includes an extracellular matrix and/or the use of normal cells obtained from the patient of interest in the region where the tumor exists.

Although the present invention has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances.

What is claimed:

1. A method of screening chemotherapeutics for second-line chemotherapy, comprising:
culturing cancer cells of interest to prepare plural first cultures;
exposing each of the first cultures to one of a plurality of first chemotherapeutic agents;
identifying a most effective member of the plurality of first chemotherapeutic agents;
culturing cancer cells that survived exposure to the most effective first chemotherapeutic agent to prepare plural second cultures;
exposing each of the second cultures of cancer cells to one of a plurality of second chemotherapeutic agents; and
identifying a most effective member of the plurality of second chemotherapeutic agents.

2. A method of screening chemotherapeutics for second-line chemotherapy, comprising:
preparing plural substrates, each substrate including a layer of normal cells on an organic matrix;
culturing cancer cells of interest on the prepared substrates to prepare plural first cultures; and
exposing each of the first cultures to one of a plurality of first chemotherapeutic agents.
